(19) United States
(12) Patent Application Publication
(10) Pub. No.: US 2025/0197338 A1
(43) Pub. Date: Jun. 19, 2025

(54) SYNTHESES OF COMPOUNDS USEFUL FOR PRODUCING TREPROSTINIL

(71) Applicant: United Therapeutics Corporation, Silver Spring, MD (US)

(72) Inventors: Hitesh Batra, Herndon, VA (US); Sri Harsha Tummala, Owings Mills, MD (US); Mark Farquharson, Vienna, VA (US)

(73) Assignee: United Therapeutics Corporation, Silver Spring, MD (US)

(21) Appl. No.: 18/941,032

(22) Filed: Nov. 8, 2024

Related U.S. Application Data
(60) Provisional application No. 63/597,473, filed on Nov. 9, 2023.

(51) Int. Cl. C07C 51/36; B01J 31/00; C07C 29/09

(52) U.S. Cl. CPC C07C 51/36; B01J 31/003; C07C 29/095; C12Y 101/01; C07C 2523/46

(57) ABSTRACT

Provided are methods of producing compounds, which are useful in synthesizing prostacyclin derivatives, such as treprostinil.

### CROSS-REFERENCE TO RELATED APPLICATIONS

The present application claims priority to U.S. Provisional Application No. 63/597,473, filed Nov. 9, 2023, which is incorporated herein by reference in its entirety.

### FIELD

The present disclosure relates to methods of producing compounds, which are useful in synthesizing prostacyclin derivatives, such as treprostinil.

### SUMMARY

One is embodiment is a method of synthesizing a compound of formula (1)

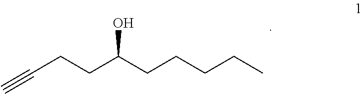

The method comprises:
reacting a compound of formula (8)

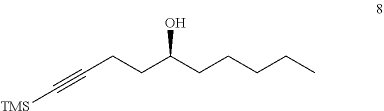

with 3,5 dinitrobenzoyl chloride to form a compound of formula (9)

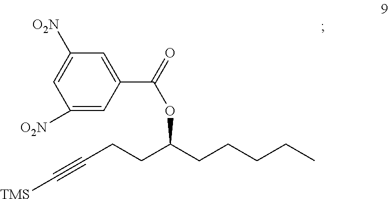 [SMILES: CCCCC[C@@H](CCC#C[Si](C)(C)C)OC(=O)c1cc([N+](=O)[O-])cc([N+](=O)[O-])c1]

and
deprotecting the compound of formula (9) to form the compound of formula (1)

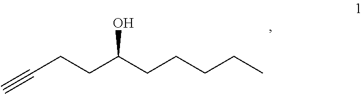

wherein TMS is trimethylsilyl.

Another embodiment is a method of synthesizing a compound of formula (1)

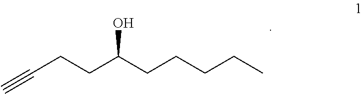

The method comprises
reacting a compound of formula (10)

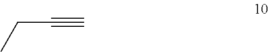

with a compound of formula (11)

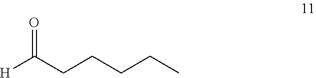

to form a compound of formula (12)

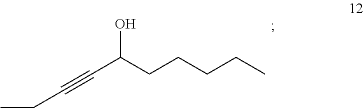

oxidizing the compound of formula (12) to form a compound of formula (13)

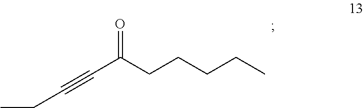

enantioselectively reducing the compound of formula (13) to form a chiral compound of formula (14)

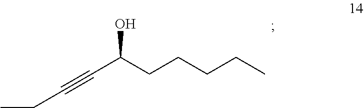

and
isomerizing the compound of formula (14) to form the compound of formula (1)

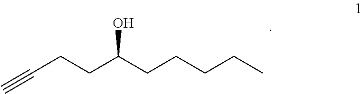

Yet another embodiments is a method of synthesizing treprostinil comprising:
enantioselectively reducing a compound of formula (16)

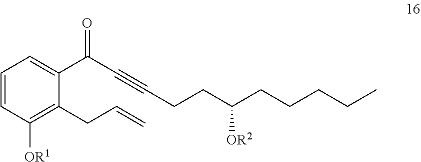

to form a compound of formula (17)

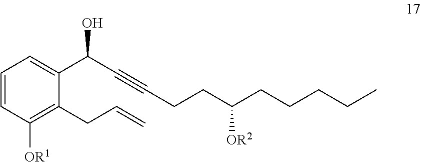

in the presence of an enantioselective enzyme catalyst; and
converting the compound of formula (17) into treprostinil (4)

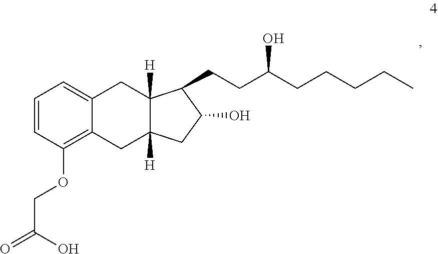

wherein R¹ is a hydroxy protecting group —(CH₂)ₙX; X being H, phenyl, —CN, —OR³ or COOR³; n being 1 to 3, R³ being an alkyl, a hydroxy protecting group or a substituted or unsubstituted benzyl group; and R² is a hydroxy protecting group.

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. Other than in the operating examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims. In order that the present disclosure can be more readily understood, certain terms are first defined. Additional definitions are set forth throughout the detailed description.

All numerical designations, e.g., pH, temperature, time, concentration, and molecular weight, including ranges, are approximations which are varied (+) or (−) by increments of 0.05%, 1%, 2%, 5%, 10% or 20%. It is to be understood, although not always explicitly stated that all numerical designations are preceded by the term “about.” It also is to be understood, although not always explicitly stated, that the reagents described herein are merely exemplary and that equivalents of such are known in the art.

As used herein, “an alcohol protecting group” or “a hydroxy protecting group” is a functional group that protects the alcohol (hydroxy) group from participating in reactions that are occurring in other parts of the molecule. Suitable alcohol protecting groups are well known to those of ordinary skill in the art and include those found in T. W. Greene, Protecting Groups in Organic Synthesis, John Wiley & Sons, Inc. 1981, or in “Greene's Protective Groups in Organic Synthesis” by Peter G. M. Wuts, Theodora W. Greene 4th edition, 2007, the entire teachings of which are incorporated herein by reference. Exemplary alcohol protecting groups include, but are not limited to, actetyl, benzoyl, benzyl, p-methoxyethoxymethyl ether, methoxymethyl ether, dimethoxytrityl, p-methoxybenzyl ether, trityl, silyl ether (e.g., trimethylsilyl (TMS), tert-butyldimethylsilyl (TBMDS), tert-butyldimethylsilyloxymethyl (TOM) or triisopropylsilyl (TIPS) ether), tetrahydropyranyl (THP), methyl ether and ethoxyethyl ether (EE).

An alkyl group may be a saturated straight-chain or branched aliphatic group. For example, an alkyl group may a (C₁-C₆)alkyl, (C₁-C₅)alkyl, (C₁-C₄)alkyl or (C₁-C₃)alkyl. Examples of alkyl groups include methyl, ethyl, n-propyl, isopropyl, n-butyl, isobutyl, sec-butyl, tert-butyl, pentyl, iso-amyl, and hexyl. An alkyl group is optionally substituted with an alkyl, a cycloalkyl (e.g., cyclopentyl or cyclohexyl), an aryl (e.g., phenyl), or heteroaryl group.

A phenyl group may be optionally substituted with one or more substituents, which may be independently selected from the group consisting of —NO₂, —CN, halogen (e.g., —F, —Cl, —Br or —I), (C₁-C₃)alkyl, halo (C₁-C₃)alkyl, (C₁-C₃)alkoxy and halo (C₁-C₃)alkoxy.

A substituted benzyl group may be optionally substituted at one or more meta, ortho or para positions with one or more substituents, which may be independently selected from the group consisting of —NO₂, —CN, halogen (e.g., —F, —Cl, —Br or —I), (C₁-C₃)alkyl, halo (C₁-C₃)alkyl, (C₁-C₃)alkoxy and halo (C₁-C₃)alkoxy.

Treprostinil, the active ingredient in Remodulin® (treprostinil) Injection, Tyvaso® (treprostinil) Inhalation Solution, and Orenitram® (treprostinil) Extended Release Tablets, was described in U.S. Pat. No. 4,306,075. Methods of making treprostinil and other prostacyclin derivatives are described, for example, in Moriarty, et al., J. Org. Chem. 2004, 69, 1890-1902, Drug of the Future, 2001, 26 (4), 364-374, U.S. Pat. Nos. 6,441,245, 6,528,688, 6,700,025, 6,809,223, 6,756,117, 8,461,393, 8,481,782; 8,242,305, 8,497,393, 8,748,657, 8,940,930, 9,029,607, 9,156,786, 9,388,154, 9,346,738; 9,593,066; 9,604,901; 10,478,410; 10,322,099; 10,548,863; 11,723,887; U.S. Published Patent Application Nos. 2012-0197041, 2013-0331593, 2014-0024856, 2015-0299091, 2015-0376106, 2016-0107973, 2015-0315114, 2016-0152548, and 2016-0175319; PCT Publication No. WO2016/0055819 and WO2016/081658.

Various uses and/or various forms of treprostinil are disclosed, for examples, in U.S. Pat. Nos. 5,153,222, 5,234,953, 6,521,212, 6,756,033, 6,803,386, 7,199,157, 6,054,486, 7,417,070, 7,384,978, 7,879,909, 8,563,614, 8,252,839, 8,536,363, 8,410,169, 8,232,316, 8,609,728, 8,350,079, 8,349,892, 7,999,007, 8,658,694, 8,653,137, 9,029,607, 8,765,813, 9,050,311, 9,199,908, 9,278,901, 8,747,897, 9,358,240, 9,339,507, 9,255,064, 9,278,902, 9,278,903, 9,758,465; 9,422,223; 9,878,972; 9,624,156; U.S. Published Patent Application Nos. 2009-0036465, 2008-0200449, 2008-0280986, 2009-0124697, 2014-0275616, 2014-0275262, 2013-0184295, 2014-0323567, 2016-0030371, 2016-0051505, 2016-0030355, 2016-0143868, 2015-0328232, 2015-0148414, 2016-0045470, 2016-0129087, 2017-0095432; 2018-0153847; 2021-0330621 and PCT Publications Nos. WO00/57701, WO20160105538, WO2016038532, WO2018/058124.

Treprostinil has the following chemical formula:

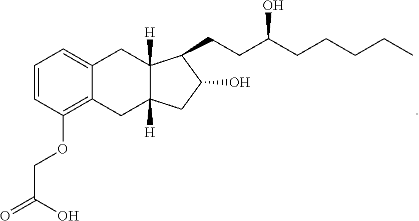 [SMILES: CCCCC[C@H](O)CC[C@@H]1[C@H]2Cc3cccc(OCC(=O)O)c3C[C@H]2C[C@H]1O]

### (A) Side Chain Synthesis

Many processes of synthesizing treprostinil, including those of U.S. Pat. Nos. 6,441,245, 6,700,025, 8,481,782, 8,940,930, use as one of starting materials side chain (1), (5S)-1-Decyn-5-ol ((S)-1-Decyne-5-ol or

(S)-dec-1-yn-5-ol):

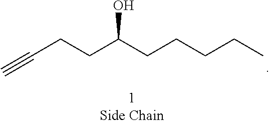

Scheme 1 illustrates a process of synthesizing treprostinil using side chain (1). Side chain (1) is converted into protected side chain (3), which has R² being a hydroxy protecting group, such as, for example, tetrahydrofuranyl (THP), benzyl, 2,4-dinitrobenzyl, methoxymethyl (MOM), tertiarybutyldimethylsilyl (TBDMS), tertiarybutyldiphenylsilyl (TBDPS), triethylsilyl (TES), benzyloxy groups, including substituted benzyloxy groups. Hydroxy protecting groups disclosed in “Greene's Protective Groups in Organic Synthesis” by Peter G. M. Wuts, Theodora W. Greene 4th addition may be used as R². Side chain (3) is reacted with aldehyde 2 having R¹ being a hydroxy protecting group and/or R¹ being —(CH₂)ₙX; X being H, phenyl, —CN, —OR³ or COOR³; n being 1 to 3, R³ being an alkyl, a hydroxy protecting group, such as THP or TBDMS, a substituted or unsubstituted benzyl group, a phenol protecting group. Hydroxy protecting groups and phenol protecting groups are disclosed in “Greene's Protective Groups in Organic Synthesis” by Peter G. M. Wuts, Theodora W. Greene 4th edition. The reacting of side chain (3) with aldehyde (2) gives racemic alcohol compound (15), which may be converted into treprostinil, for example, as disclosed in U.S. Pat. Nos. 6,441,245, 6,700,025, 8,481,782, 8,940,930 or the process discussed in this disclosure.

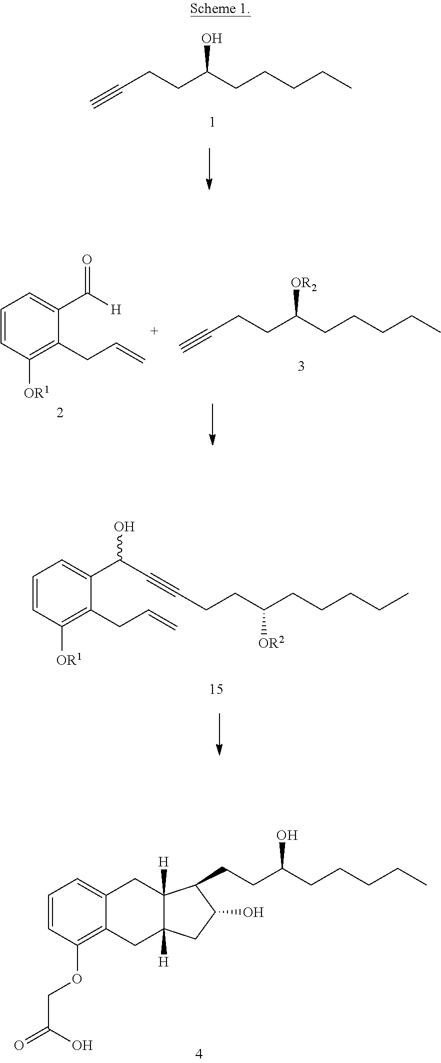

The current synthesis of side chain (1) is illustrated in Scheme 2.

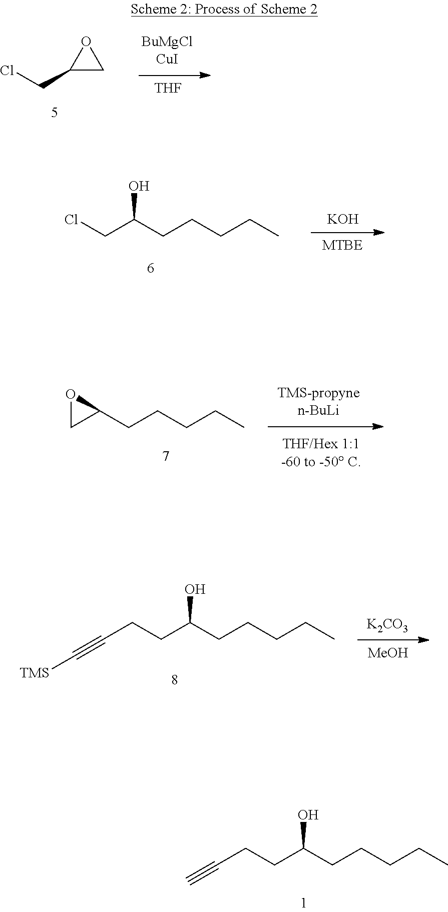

The synthesis process of Scheme 2 may have a number of drawbacks. For example, this process uses expensive reagents, such as chiral S-epichlorohydrin and TMS-propyne, which add to the overall cost of the process. The process of Scheme 2 uses a deprotection step. The process of Scheme 2 generates difficult-to-remove isomeric impurities leading to tedious column chromatographic purifications. Typically, ˜1500 to 1600 L of solvent(s) may be needed for the purification of per kilogram (kg) of side chain compound (1) and a second column purification may be also needed on impure fractions to improve the yields. This large excess of solvents adds to the cost of the process as well as to the cost of disposal. The overall yield of the process is approximately 31%.

The present disclosure provides two alternative processes: Process of Scheme 3 and Process of Scheme 4, for synthesizing side chain (1). These alternative processes may overcome at least some of the discussed above drawbacks of the process of Scheme 2. Table 1 provides comparison of Process of Scheme 2, Process of Scheme 3 and Process of Scheme 4.

TABLE 1

| | Process of | Process of | Process of |
|---|---|---|---|
| Attribute | Scheme 2 | Scheme 3 | Scheme 4 |
| Number of Steps | 4 | 5 | 4 |
| Solvent Volume for | ~1500-1600 L | ~550-650 L* | ~400-500 L* |
| purification of 1 kg | | | |
| of side chain (1) | | | |
| % Solvent reduction by | Not applicable | ~63% | ~73% |
| conservative estimates | | | |
| Purity by GC | ≥97% | ≥97% | ≥97% |
| Chiral Purity by GC | ≥98% | ≥98% | ≥98% |
| Overall yield | ~31% | ~43% | ~56% |
| Yield Increase | Not applicable | ~39% | ~81% |

*Calculated volume based on lab work

Process of Scheme 3 for synthesizing the side chain compound of formula (1) may involve reacting TMS alkyne compound of formula (8)

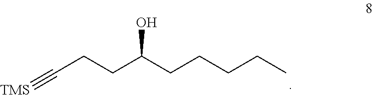

(TMS is trimethylsilyl) with 3,5 dinitrobenzoyl chloride to form 3,5 dinitrobenzate compound of formula

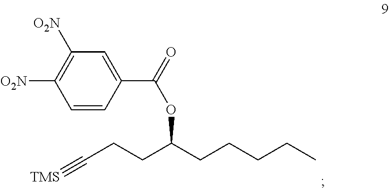

and deprotecting the compound of formula (9) to form the compound of formula (1)

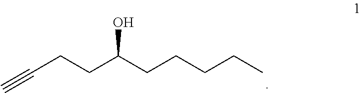

The TMS alkyne compound of formula (8) may prepared by (A) reacting a compound of formula (5)

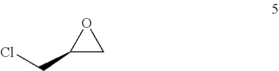

((S)-epichorohydrin) with butylmagnesium halide, such as butylmagnesium chloride, to form a compound of formula (6)

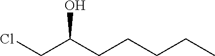

(chloroheptanol); (B) reacting the compound of formula (6) with a base to form a compound of formula (7)

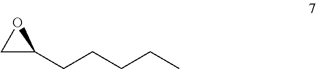

(epoxyheptane); and (C) reacting the compound of formula (7) with trimethylsilyl propyne to form the compound of formula (8).

Scheme 3 below illustrates the reactions of process (1) starting with(S)-epichorohydrin. Specific reaction conditions are listed in Scheme 3 only for illustrative purposes.

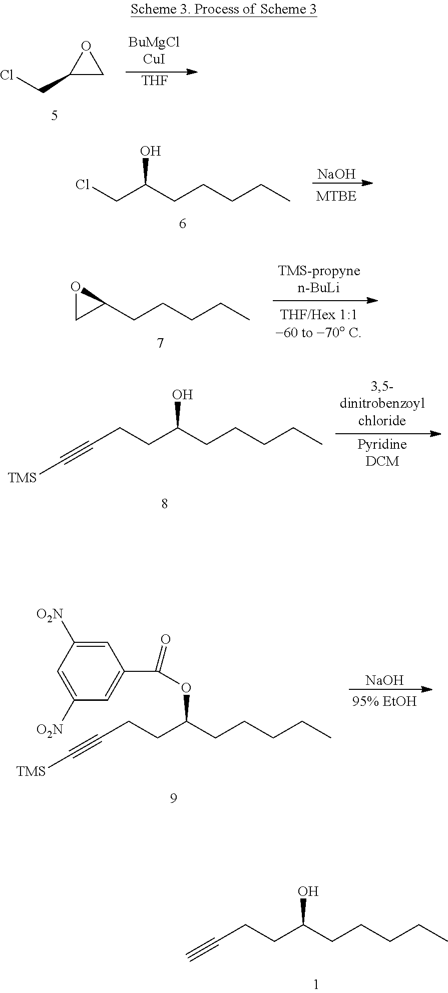

The first three reactions in Scheme 3 are similar to the first three reactions in Scheme 2. Thus, in some embodiments, the TMS alkyne compound of formula (8) for Process of Scheme 3 may be obtained using the first three reactions in Scheme 2.

In the first reaction in Scheme 3, the compound of formula (5) ((S)-epichorohydrin) is reacted with butylmagnesium halide, such as butylmagnesium chloride, to form the compound of formula (6) (chloroheptanol). This reaction may be performed in the presence of a reagent, such as CuI. A number of solvents may be used for this reaction, including tetrahydrofuran (THF), t-butyl methylether (MTBE), 2-methyl tetrahydrofuran (2-MeTHF), diethyl ether and their mixtures.

In the second reaction in Scheme 3, the compound of formula (6) is reacted with a base, such as NaOH or KOH, to form a compound of formula (7) (epoxyheptane). The base may be an or any inorganic base, such as NaOH or KOH, or an organic base. A number of solvents may be used for this reaction, including methyl tert-butyl ether (MTBE) and tert-amyl ethyl ether (TAEE), water, tetrahydrofuran, dichloromethane, methanol, acetonitrile, toluene, dimethylformamide, diethyl ether, hexane, ethanol, pyridine, 1,4-dioxane, acetone, isopropanol, tert-butanol, dimethyl sulfoxide, benzene, 1,2-dichloroethane, ethyl acetate and their mixtures.

In the third reaction in Scheme 3, the compound of formula (7) is reacted with trimethylsilyl propyne to form TMS alkyne compound of formula (8). This reaction may be performed in the presence of a base, such as an organolithium base, e.g. n-Butyllithium (abbreviated n-BuLi) or reacting with Grignard reagent of alkynyl bromide, such as 3-Bromo-1-(trimethylsilyl)-1-propyne. The reaction may be performed at a temperature from −75° C. to −45° C. or from −70° C. to −60° C. or from −60° C. to −50° C. A number of solvents may be used for this reaction, including THF, hexane, t-butyl methylether (MTBE), 2-methyl tetrahydrofuran (2-MeTHF), diethyl ether, or their mixtures.

In the fourth reaction in Scheme 3, the TMS alkyne compound of formula (8) is reacted with 3,5 dinitrobenzoyl chloride to derivatize the hydroxy group of the compound of formula (8) and thereby, form 3,5 dinitrobenzate compound of formula (9). This reaction may be performed in the presence of a base. In some embodiments, the base a organic base, such as pyridine, triethylamine, diisopropylethyl amine, 1,8-Diazabicyclo[5.4.0]undec-7-ene (DBU). Yet in some embodiments, the base may be an inorganic base, such as ammonium hydroxide or cesium carbonate. A number of solvents may be used for this reaction, including dicholoromethane (DCM), ethyl acetate, tetrahydrofuran, diethyl ether, t-butyl methylether (MTBE), 2-methyl tetrahydrofuran, dicholorethane, and their mixtures.

In the fifth reaction in Scheme 3, the compound of formula (9) is deprotected to form the compound of formula (1). The deprotection reaction may be performed in the presence of a base. The base may be an inorganic base, such as NaOH or KOH, or an organic base. A number of solvents may be used for this reaction, including a lower alcohol, such as propanol, ethanol, or methanol, or its mixture with water.

The five reactions of Scheme 3 may consume no more than 1000 L or no more than 900 L or not more than 800 L or no more than 750 L or no more than 600 L or no more than 650 L or from about 550 L to about 650 L or solvents for purification by column chromatography for per 1 kg of the compound of formula (1).

A chiral purity of the product of the five reactions of Scheme 3, which product contain the compound of formula (1), may be at least 97.5% or at least 97.8% or at least 98% or at least 98.1% or at least 98.2% or at least 98.3% or at least 98.4% or at least 98.5% or at least 98.6% or at least 98.7% or at least 98.8% or at least 98.9% or at least 99.0% or at least 99.1% or at least 99.2% or at least 99.3% or at least 99.4% or at least 99.5% or at least 99.6% or at least 99.7% or at least 99.8% or at least 99.9%. The chiral purity may be determined, for example, by chiral gas chromatography (GC).

A chemical purity of the product of the five reactions of Scheme 3, which product contain the compound of formula (1), may be at least 97.5% or at least 97.8% or at least 98% or at least 98.1% or at least 98.2% or at least 98.3% or at least 98.4% or at least 98.5% or at least 98.6%.

Process of Scheme 4 for synthesizing the side chain compound of formula (1) may involve reacting a compound of formula (10)

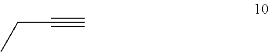

(1-butyne) with an aldehyde compound of formula (11)

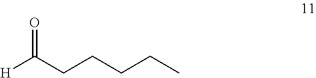

to form a racemic propargylic alcohol compound of formula (12)

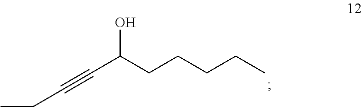

oxidizing the compound of formula (12) to form a ynone compound of formula (13)

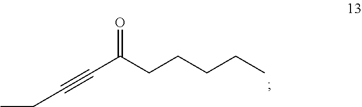

enantioselectively reducing the compound of formula (13) to form a chiral propargylic alcohol compound of formula (14)

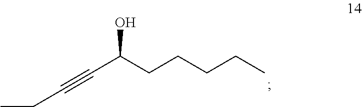

and isomerizing the compound of formula (14) to form the compound of formula (1).

Scheme 4 below illustrates the reactions of process of Scheme 4. Specific reaction conditions are listed in Scheme 4 only for illustrative purposes.

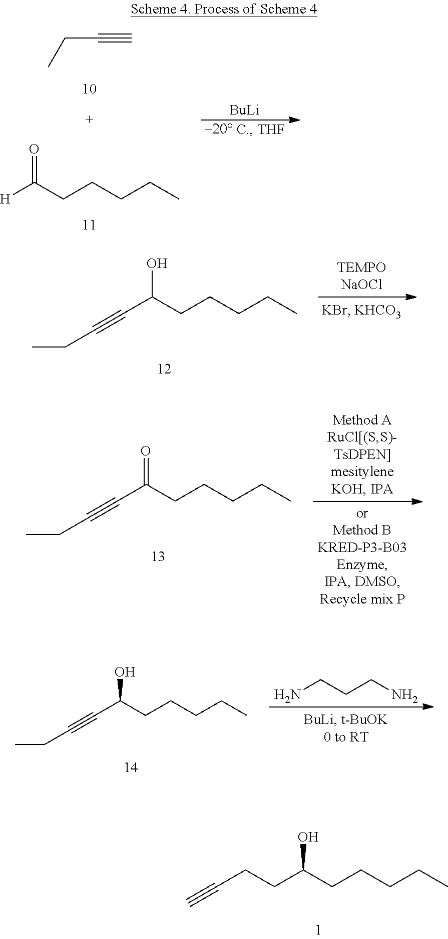

In the first reaction in Scheme 4, the compound of formula (10) (1-butyne) is reacted with the aldehyde compound of formula (11) to form the racemic propargylic alcohol compound of formula (12). This reaction may be performed in the presence of a base, such as an organolithium base, e.g. n-Butyllithium (abbreviated n-BuLi). The reaction may be performed at a temperature from −78° C. to 20° C. or from −50° C. to −5° C. or from −25° C. to −15° C., such as about-20° C. A number of solvents may be used for this reaction, including THF, t-butyl methylether (MTBE), 2-methyl tetrahydrofuran (2Me-THF), diethyl ether, and their mixtures.

In the second reaction in Scheme 4, the compound of formula (12) is oxidized to form a ynone compound of formula (13). This reaction may be performed in the presence of an oxidation catalyst, such as (2,2,6,6-Tetramethylpiperidin-1-yl)oxyl or an oxidizing agents, such as chromium trioxide, manganese dioxide, hypervalent iodine based reagents,

Tetrapropylammonium perruthenate, or Swern oxidizing reagent. The reaction may be performed in the presence of one or more salts, such as NaOCl, KBr and KHCO₃. A number of solvents may be used for this reaction, including DCM, ethyl acetate, dichloroethane, tetrahydrofuran and their mixtures.

In the third reaction of Scheme 4, the compound of formula (13) is enantioselectivity reduced to form a chiral propargylic alcohol compound of formula (14).

The enantioselective reduction may be performed in the presence of an enantioselective catalyst or an enantioselective reducing agent.

In some embodiments, the enantioselective catalyst may be a metal containing catalyst, such as a transitional metal containing catalyst. For example, in some embodiments, the metal containing catalyst may be a preformed ruthenium containing catalyst, such as RuCl[(S,S)-TsDPEN](p-cymene) (also known as [(S,S)—N-(2-Amino-1,2-diphenylethyl)-p-toluenesulfonamide]chloro(p-cymene)ruthenium (II)) or RuCl[(S,S)-TsDPEN](mesitylene) (also known as [N-[(1S,2S)-2-(Amino-κN)-1,2-diphenylethyl]-4-methylbenzenesulfonamidato-κN]chloro[(1,2,3,4,5,6-n)-1,3,5-trimethylbenzene]-ruthenium). The catalyst may also be generated in situ using dichloro(mesitylene) ruthenium (II) dimer and (1S,2S)-(+)-N-p-Tosyl-1,2-diphenylethylenediamine ligand or dichloro(p-cymene)ruthenium (II) dimer and (1S,2S)-(+)-N-p-Tosyl-1,2-diphenylethylenediamine ligand. Other reducing agents include borane reagents, such as catecholborane; lithium based reagents, such as lithium aluminumhydride; sodium based reagents, such as sodium borohydride, in combination with a chiral ligand, such as 3-nitrophenylboronicacid-L-tartaric acid ester.

The enantioselective reduction in the presence of a ruthenium containing catalyst, such as RuCl[(S,S)-TsDPEN](mesitylene), may be performed also in the presence of a base, such as NaOH or KOH or triethylamine: formic acid or any other hydrogen source. A number of solvents may be used for this reaction, including isopropanol (IPA), dichloromethane and their mixture.

In some embodiments, the enantioselective catalyst may be an enzyme catalyst. In some embodiments, the enzyme catalyst may be a ketoreductase, such as a ketoreductase derived from the Thermoanaerobacter brockii alcohol dehydrogenase, e.g. KRED-P3-B03 or KRED-P2-G03 or KRED-130. Ketoreductase compounds, such as KRED-P3-B03, are commercially available from Codexis, Inc, California, U.S.A.

The enantioselective reduction in the presence of an enzyme catalyst, such as a ketoreductase, e.g. KRED-P3-B03, may be performed in the presence of reconstituted KRED Recycle Mix P which may contain sodium phosphate, magnesium sulfate, and nicotinamide adenine dinucleotide phosphate (NADP⁺). KRED Recycle Mix P may have a pH of about 7.0. In one exemplary embodiment, a reconstituted KRED Recycle Mix P may contain 128 mM sodium phosphate, 1.7 mM magnesium sulfate, 1.1 mM NADP⁺, pH 7.0 per 250 mg of an enzyme catalyst, such as a ketoreductase, e.g. KRED-P3-B03. A number of solvents may be used for this reaction, including IPA, dimethylsulfoxide (DMSO) and their mixtures.

In the fourth reaction in Scheme 4, the compound of formula (14) is isomerized to form the compound of formula (1). The isomerization reaction may be a zipper isomerization reaction.

The isomerization reaction may be performed in the presence of a zipper isomerization reagent. In some embodiments, the zipper isomerization catalyst may be 1,3-diaminopropane, a mixture of 1,3-diaminopropane and alkali metal compound (e.g. a lithium compound, such as lithium metal, BuLi, or a potassium compound, such as potassium hydride or potassium tert-butoxide, or a sodium compound, such as sodium hydride) or an alkali metal salt of 1,3-diaminopropane, such as a lithium, sodium or potassium salt of 1,3-diaminopropane. For example, in one embodiment, the isomerization reaction may be performed in the presence of 1,3-diaminopropane, butyl lithium and potassium tert-butoxide.

A number of solvents may be used in the isomerization reaction, including THF, 1,3-diaminopropane, 2-methyl tetrahydrofuran (2Me-THF), t-butyl methylether (MTBE), hexane, heptane, diethyl ether, and their mixtures.

The four reactions of Scheme 4 may consume no more than 800 L or no more than 700 L or not more than 600 L or no more than 550 L or no more than 500 L or from about 400 L to about 500 L or solvents for purification by column chromatography of per 1 kg of the compound of formula (1).

A chiral purity of the product of the four reactions of Scheme 4, which product contain the compound of formula (1), may be at least 97.5% or at least 97.8% or at least 98% or at least 98.1% or at least 98.2% or at least 98.3% or at least 98.4% or at least 98.5%. The chiral purity may be determined, for example, by a chiral chromatography, such as chiral gas chromatography (GC).

A chemical purity of the product of the four reactions of Scheme 4, which product contain the compound of formula (1), may be at least 96.0% or at least 96.5% or at least 96.6% or at least 96.7% or at least 96.8% or at least 96.9% or at least 97.0%.

The compound of formula (1) synthesized according to Scheme 3 or 4 may be used for synthesizing treprostinil by protecting the compound of formula (1) to form a compound of formula (3)

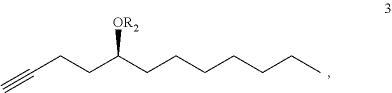

reacting the compound of formula (3) with a compound of formula (2)

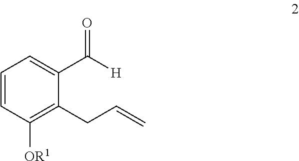

to form a compound of formula (15)

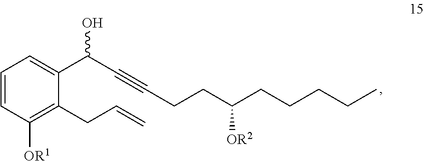

and converting the compound of formula (15) into treprostinil (4)

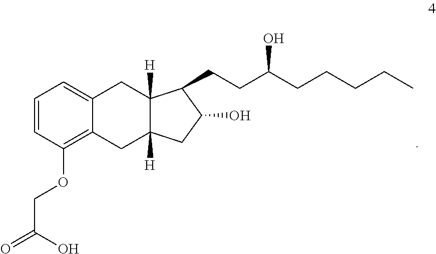 [SMILES: CCCCC[C@H](O)CC[C@@H]1[C@H]2Cc3cccc(OCC(=O)O)c3C[C@H]2C[C@H]1O]

R¹ and R² may be as defined above. The reacting the compound of formula (3) with the compound of formula (2) to form the compound of formula (15) and the converting the compound of formula (15) into treprostinil may be performed as disclosed in U.S. Pat. Nos. 6,441,245, 6,700,025, 8,481,782, 8,940,930.

### Treprostinil Synthesis

Many current treprostinil synthesis processes, including those disclosed in U.S. Pat. Nos. 6,441,245, 6,700,025, 8,481,782, 8,940,930, use the following enantioselective reduction of aryl alkynyl ketone (16) to obtain chiral alcohol (17), which may be later converted into treprostinil.

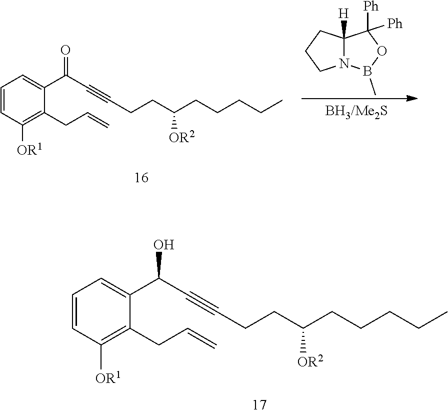

Aryl alkynyl ketone (16) for this reaction may be obtained from racemic alcohol (15), see Scheme 1, using, for example, Pyridinium chlorochromate (PCC) oxidation reaction as disclosed, for example, in U.S. Pat. Nos. 6,441,245, 6,700,025, 8,481,782, 8,940,930. The enantioselective reduction reaction of aryl alkynyl ketone (16) to obtain chiral alcohol (17) uses a R-CBS reagent, such as (R)-2-Methyl-CBS-oxazaborolidine. Scheme 5 below illustrates the process of synthesizing treprostinil disclosed in U.S. Pat. No. 6,441,245, which uses the enantioselective reduction reaction of aryl alkynyl ketone (16) to obtain chiral alcohol (17) uses a R-CBS reagent.

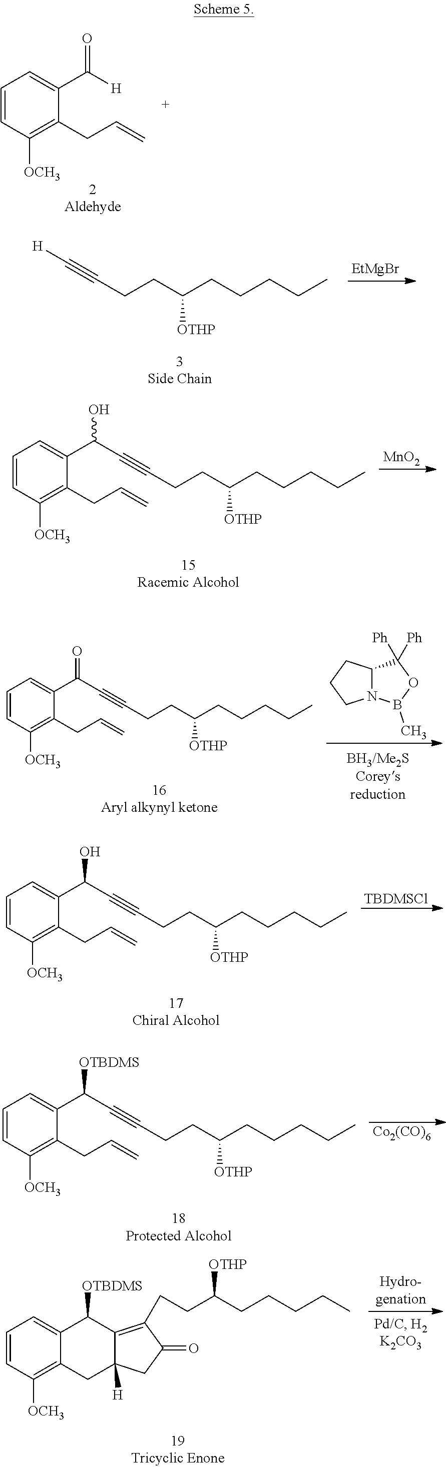

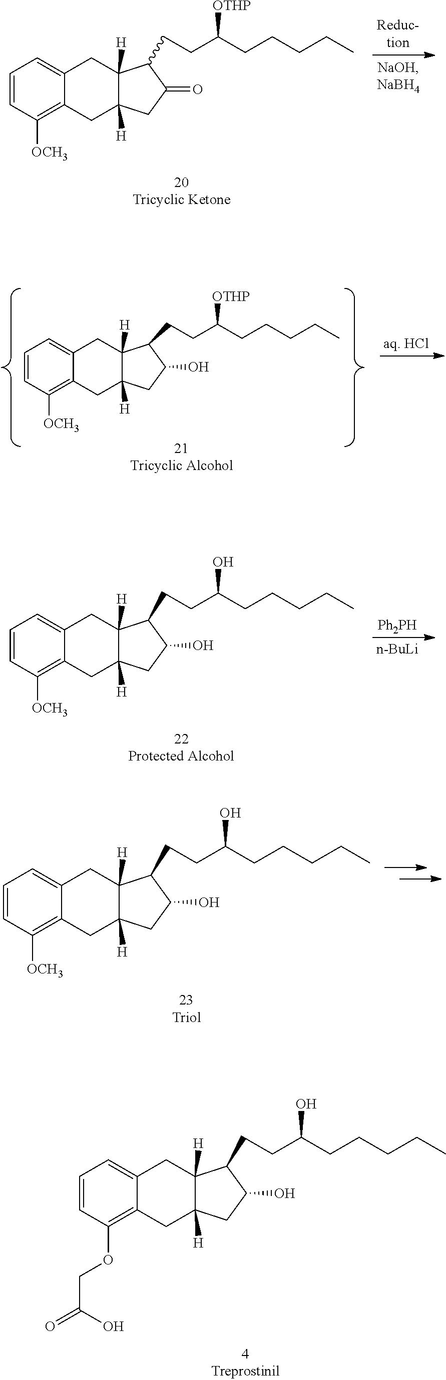

The enantioselective reduction reaction of aryl alkynyl ketone (16) using R-CBS reagent(s) may have a number of drawbacks. For example, this reaction yields typically only ˜95-96% chiral selectivity. The reduced chiral selectivity leads to a known impurity (3AU90 isomer) in the process. Tedious column chromatography to remove borates and other by-products may be needed to isolate the product. Cryogenic conditions may be needed for the reaction. The borane-dimethyl sulfide complex typically used with R-CBS reagent(s) in treprostinil synthesis may have an unpleasant, obnoxious smell.

The present disclosure provides an alternative enantioselective reduction reaction of aryl alkynyl ketone (16) to produce chiral alcohol (17), which may avoid some or all of the drawbacks of the enantioselective reduction reaction of aryl alkynyl ketone (16) using R-CBS reagent(s).

A method of synthesizing treprostinil may involve
enantioselective reduction of a aryl alkynyl ketone compound of formula (16)

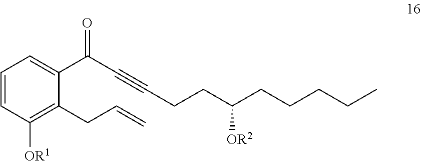

to form a chiral alcohol compound of formula (17)

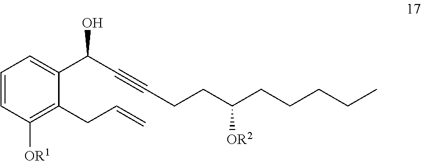

in the presence of an enantioselective enzyme catalyst; and
converting the compound of formula (17) into treprostinil (4)

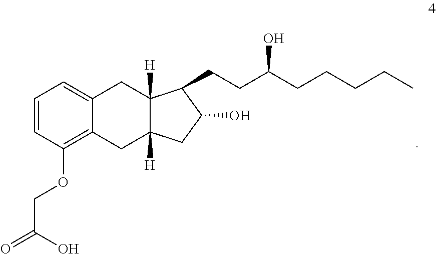 [SMILES: CCCCC[C@H](O)CC[C@@H]1[C@H]2Cc3cccc(OCC(=O)O)c3C[C@H]2C[C@H]1O]

The converting the compound of formula (15) into treprostinil may be performed as disclosed in U.S. Pat. Nos. 6,441,245, 6,700,025, 8,481,782, 8,940,930.

The enantioselective reduction of the compound of formula (16) to form the compound of formula (17) in the presence of an enantioselective enzyme catalyst is illustrated in Scheme 6. Specific reaction conditions are listed in Scheme 6 only for illustrative purposes.

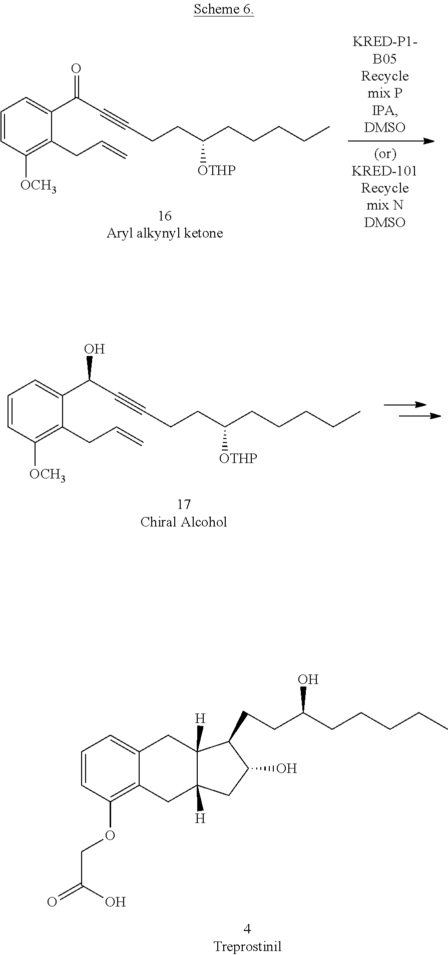

In some embodiments, the enantioselective enzyme catalyst may be a ketoreductase, such as KRED-P1-B05 or KRED-101. Ketoreductase compounds, such as KRED-P1-B05 or KRED-101, are commercially available from Codexis, Inc, California, U.S.A.

In some embodiments, the enantioselective reduction in the presence of an enzyme catalyst, such as a ketoreductase, e.g. KRED-P3-B05, may be performed in the presence of reconstituted KRED Recycle Mix P which may contain sodium phosphate, magnesium sulfate, and nicotinamide adenine dinucleotide phosphate (NADP+). The KRED Recycle Mix P may have a pH of about 7.0. In one exemplary embodiment, a reconstituted KRED Recycle Mix P may contain 128 mM sodium phosphate, 1.7 mM magnesium sulfate, 1.1 mM NADP⁺, pH 7.0 per 250 mg of an enzyme catalyst, such as a ketoreductase, e.g. KRED-P3-B05.

In some embodiments, the enantioselective reduction in the presence of an enzyme catalyst, such as a ketoreductase, e.g. KRED-10, may be performed in the presence of reconstituted KRED Recycle Mix N contains sodium phosphate, magnesium sulfate, NADP⁺, nicotinamide adenine dinucleotide (NAD⁺), D-glucose, and glucose dehydrogenase. The KRED Recycle Mix N may have a pH of about 7.0. In one exemplary embodiment, reconstituted KRED Recycle Mix N contains 263 mM sodium phosphate, 1.7 mM magnesium sulfate, 1.1 mM NADP⁺, 1.1 mM NAD⁺, 80 mM D-glucose, 4.3 U/mL glucose dehydrogenase, pH 7.0.per 250 mg of an enzyme catalyst, such as a ketoreductase, e.g. KRED-101.

A number of solvents may be used for the enantioselective reduction reaction, including IPA, dimethylsulfoxide (DMSO) and their mixtures.

In some embodiments, a product of the enantioselective reduction performed in the presence of an enantioselective enzyme catalyst, which product contains the compound of formula (17); may have a chiral purity of at least 98%, or at least 98.1%, or at least 98.2%, or at least 98.3% or at least 98.4% or at least 98.5%, or at least 98.6% or at least 98.7% or at least 98.8% or at 98.9% or at least 99%. The chiral purity may be determined, for example, by chiral chromatography, such as chiral gas chromatography (GC) or chiral high-performance liquid chromatography (HPLC).

The chiral purity of the product of the enantioselective reduction performed in the presence of an enantioselective enzyme catalyst may be higher than a chiral purity of a product of a enantioselective reduction performed in the presence of R-CBS reagents, such as the enantioselective reactions in U.S. Pat. Nos. 6,441,245, 6,700,025, 8,481,782, 8,940,930. Because the higher chiral purity for the enantioselective reduction performed in the presence of an enantioselective enzyme catalyst, a final treprostinil batch produced by a process including such enantioselective reduction may have a lower concentration of impurities, such as 3AU90 isomer, compared a treprostinil batch produced by a process including a enantioselective reduction performed in the presence of R-CBS reagents, such as the processes in U.S. Pat. Nos. 6,441,245, 6,700,025, 8,481,782, 8,940,930. For example, in some embodiments a content of a 3AU90 isomer in a treprostinil batch synthesized by a process including the enantioselective reduction performed in the presence of an enantioselective enzyme catalyst may be 0.3 mass % or less, 0.25 mass % or less, 0.2 mass % or less, 0.15 mass % or less, 0.1 mass % or less or 0.05 mass % or less.

Embodiments described herein are further illustrated by, though in no way limited to, the following working examples.

### Example 1: Process of Scheme 2 for Side Chain Synthesis

### Step 1: Synthesis of Chloroheptanol (6)

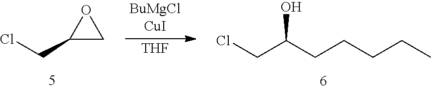

A 2000 mL 3-neck round bottom flask was charged with a solution of(S)-epichlorohydrin (5) (100.0 g, 1.08 mol) in anhydrous THF (500 mL) and cupper iodide (20.95 g, 1.13 mol) was added. The reaction mixture was stirred for 0.5 h and cooled to 0-5° C. under argon. Then butylmagnesium chloride (609 mL) was added dropwise within 2 h. After completion, the mixture was allowed to RT and stirred overnight. The reaction was quenched with 15% NH₄OH at 0-5° C. and filtered through a celite pad and washed the pad with MTBE. The organic layer and washed were combined and washed with 15% NH₄OH, brine, then dried over Na₂SO₄. The mixture was concentrated in vacuo to obtain crude chloroheptanol (6) (183 g). The crude product was used for the next step reaction without purification. The product was characterized by ¹H NMR.

### Step 2: Synthesis of Epoxyheptane (7)

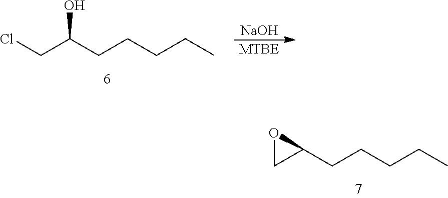

A 2000 mL 2-neck round bottom flask was charged with a solution of chloroheptanol (6) (192.0 g, 1.11 mol) in MTBE (550 mL) and was cooled to 0-5° C., then sodium hydroxide (106.4 g, 2.66 mol) was added in 4 portions within 2 h and then warmed to RT under argon. The reaction mixture was stirred overnight. The mixture was filtered through a celite pad (80 g) and washed the pad with MTBE. The filtrate was washed with brine and dried over Na₂SO₄. The mixture was concentrated in vacuo to obtain crude epoxyheptane (7) (125 g) as pale-yellow liquid. The crude epoxyheptane (7) was used for the next step reaction without purification. The product was characterized by ¹H NMR.

### Step 3: Synthesis of TMS Alkyne (8)

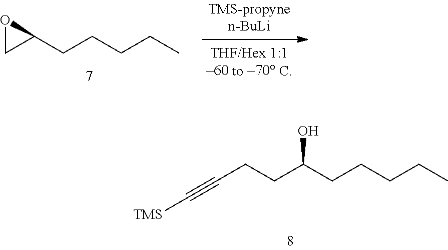

A 2000 mL reaction vessel was charged with a solution of TMS propyne (112.2.0 g, 1.0 mol) and epoxyheptane (7) (115 g, 0.77 mol) in anhydrous THF/hexane (1:1, 600 mL), then it was stirred for 10 min and was cooled to −60 to −70° C. To this n-butyllithium (2.5 M in hexane, 400 mL, 1.0 mol) was added dropwise under argon. The reaction mixture was stirred at −60 to −70° C. for 10 h and the reaction mixture was allowed to be warmed to RT and stirred overnight under argon. The reaction mixture was cooled to around 0° C. and was quenched by saturated NH₄Cl (150 mL). The reaction was stirred at RT for 0.5 h, organic layer was separated, and the aqueous layer was extracted with MTBE (2×100 mL). The combined organic layers were washed with brine, dried over Na₂SO₄, and concentrated in vacuo to provide crude TMS alkyne (8) (175.0 g) as brown liquid. The crude product was purified by silica gel column chromatography obtain pure TMS alkyne (8) (122.5 g, yield 70.2%). The product was characterized by ¹H NMR.

### Process of Scheme 3: Step 4: Synthesis of 3,5-dinitrobenzoate (9)

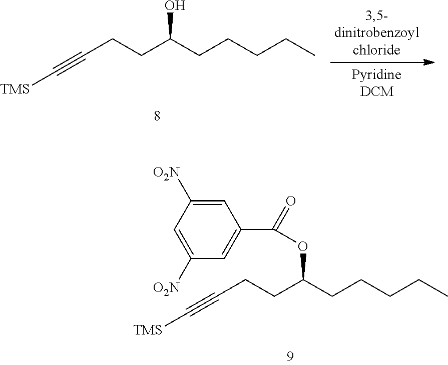 [SMILES: CCCCC[C@H](O)CCC#C[Si](C)(C)C ; CCCCC[C@@H](CCC#C[Si](C)(C)C)OC(=O)c1cc([N+](=O)[O-])cc([N+](=O)[O-])c1]

A 3000 mL reaction vessel was charged with a solution of crude TMS alkyne (8) (121.3 g) and 3,5-dinitrobenzoyl chloride (228.7 g, 0.99 mol) in anhydrous DCM (1200 mL) and was stirred for 10 min under argon at 0-5° C. To this pyridine (120 mL, 1.50 mol) was added dropwise under argon. The reaction mixture was stirred at 0-10° C. for 0.5 h and warmed to RT and stirred overnight. The reaction progress was monitored by TLC and after complete reaction, MTBE was added to reaction mixture and stirred for 0.5 h. The precipitated solids were filtered, and the solids were washed with MTBE. The filtrate was washed with saturated aq. NaHCO₃, brine and dried over Na₂SO₄. The mixture was concentrated in vacuo to provide crude 3,5 dinitrobenzoate (9) (236.2 g) as a pale-yellow solid which was crystallized from ethanol to provide pure dinitrobenzoate (184.0 g). The mother liquid was concentrated to around 500 mL and the solids was collected and washed with isopropyl alcohol (300 mL) to obtain product (30.0 g) which was slurried in hexane to provide additional product (22.5 g). The compound was characterized by 1H NMR, ¹³C NMR, and UPLC.

### Step 5: Synthesis of Side Chain (1)

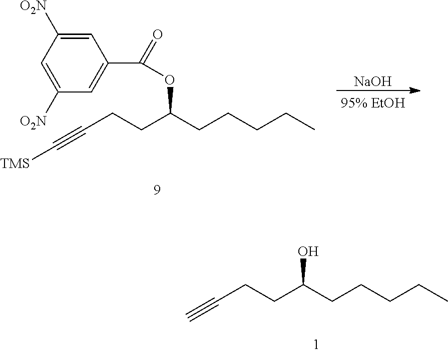

A 2000 mL round bottom flask was charged with sodium hydroxide (39.2 g, 0.98 mol) and 95% ethanol (1200 mL) and cooled to 0-5° C. and then 3,5-dinitrobenzoate (9) (206.0 g, 0.49 mol) was added under argon. The mixture was stirred at 0-5° C. for 1 h and then at RT for 4 h until the reaction was complete. At this stage MTBE was added and the mixture was filtered through a celite pad and washed with MTBE. The filtrate was concentrated in vacuo and the residue was dissolved in MTBE, washed with saturated aq. NaHCO₃, brine, dried over Na₂SO₄ and concentrated in vacuo to provide side chain (1) 71.5 g (94.7% yield). The chemical and chiral purity by GC was found to be 98.6% and 100% respectively. The compound was characterized by ¹H NMR, ¹³C NMR and IR. The overall yield over five steps was found to be 43%.

### Example 2: Process of Scheme 4 for Side Chain Synthesis (Via Enantioselective Reduction)

### Step 1: Addition of 1-Butyne (10) to Aldehyde (11)

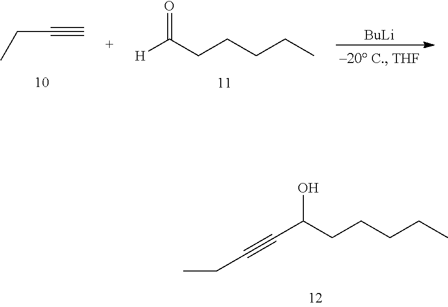

To THF (200 mL) at −20° C., 1-butyne (10) gas (13 g, 240.5 mmol) was bubbled. To this solution 2.5 M n-butyllithium in hexane (40 mL, 99.8 mmol) was added dropwise and after complete addition more butyne gas (4.88 g, 90.2 mmol) was bubbled to the reaction mixture. A solution of aldehyde (11) (10 g, 99.8 mmol) in THF was added dropwise. After complete addition the reaction mixture was allowed to warm to room temperature. The reaction was monitored by TLC and found to be complete after 2 h. The reaction was quenched with saturated ammonium chloride solution and the organic layer was extracted with MTBE. The combined organic layers were washed with water, brine, dried over sodium sulfate and evaporated in vacuo to obtain crude racemic propargylic alcohol (12) (14.7 g) which was carried to next step. The product was characterized by ¹H NMR and ¹³C NMR.

### Step 2: Oxidation of Racemic Propargylic Alcohol (12) to Ynone (13)

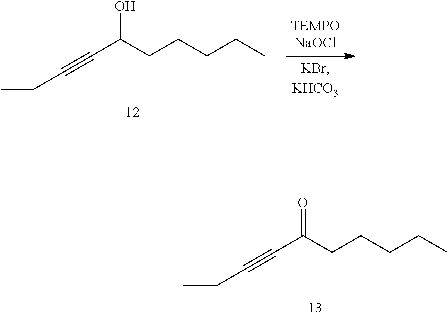

The racemic propargylic alcohol (12) (5.11 g) was dissolved in dichloromethane (100 mL) and cooled to 0° C. To this solution potassium bromide (0.78 g, 6.62 mmol) in water (13.2 mL), 2,2,6,6-tetramethylpiperidine 1-oxyl (TEMPO) (0.79 mg, 6.62 mmol) and potassium bicarbonate (41.4 g, 414 mmol) in water (138 mL) were added. A solution of bleach (10-15% by wt.) (62 mL) in water (205 mL) was added dropwise. After 3 h, the reaction was found to be complete by TLC. At this stage the organic layer was separated, and aqueous layer was extracted twice with dichloromethane. The combined organic layers were washed with 10% aq. sodium thiosulfate solution, 0.5 N HCl solution, saturated aqueous sodium bicarbonate solution, water, brine, dried over sodium sulfate and evaporated in vacuo to obtain crude ynone (13) (4.82 g). The product was characterized by ¹H NMR and ¹³C NMR.

### Step 3: Enantioselective Reduction of Ynone (13) to Chiral Propargylic Alcohol (14)

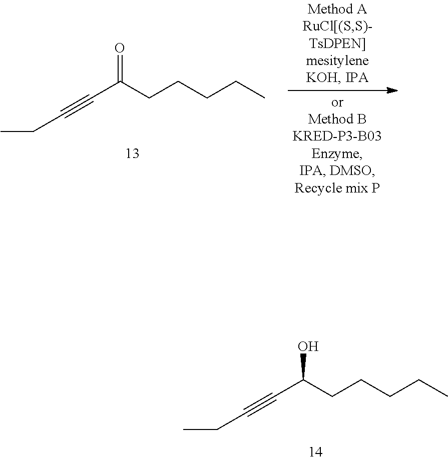

### Method a (Metal Catalyst Based Enantioselective Reduction)

A solution of ynone (13) (6.5 g, 42.70 mmol) in IPA (124 mL) was evacuated and replaced with argon (three times). To the above solution RuCl[(S,S)-TsDPEN](mesitylene) (1.06 g, 1.71 mmol) and a solution of potassium hydroxide (192 mg, 3.42 mmol) in IPA (6 mL) were added. After 2.5 h, TLC indicated completion of the reaction. The reaction mixture was evaporated in vacuo and the residue was partitioned between MTBE and 1N HCl. The organic layer was separated, and the solids were filtered through celite. The filtrate was washed with saturated sodium bicarbonate solution, water, brine, dried over sodium sulfate and evaporated in vacuo to obtain crude chiral propargylic alcohol (14) (6.33 g). The product was characterized by ¹H NMR and ¹³C NMR.

### Method B (Enzyme Based Enantioselective Reduction)

To KRED enzyme KRED-P3-B03 (10 mg) was added KRED Recycle Mix P (475 μL). A solution of ynone (13) (2 mg) in 25 μL DMSO was added. The mixture was stirred at 30° C. for 24 h. At this stage the reaction was quenched by adding MTBE (1.5 mL). The reaction mixture was centrifuged, organic layer was separated, and aqueous layer was extracted with MTBE. The combined organic layers were evaporated in vacuo to obtain crude propargylic alcohol (14) (2 mg). The chiral purity by GC was found to be 97.4%.

### Step 4: Zipper Isomerization of Chiral Propargylic Alcohol (14) to Side Chain (1)

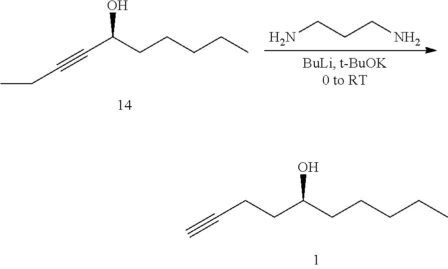

To 1,3-diaminopropane (3.3 mL, 38.90 mmol) was added THF (25 mL) under argon atmosphere and was cooled to 0° C. A solution of 2.5M n-butyllithium (10.4 mL, 25.92 mmol) was added dropwise to the reaction mixture and stirred for 40 min allowing to RT. The potassium tert-butoxide (2.9 g, 25.92 mmol) was added at RT and stirred for 30 mins. To this reaction mixture chiral propargylic alcohol (14) (1.0 g 6.48 mmol) obtained via enantioselective reduction (Method A) was added as a solution in THF (2 mL). After 2 h, TLC indicated completion of the reaction and the reaction was quenched by adding saturated aqueous ammonium chloride solution, The reaction mixture was partitioned between water and MTBE. The organic layer was separated, and the aqueous layer was extracted three times with MTBE. The combined organic layers were evaporated in vacuo. The residue was dissolved in MTBE, washed with 1N HCl, saturated aqueous sodium bicarbonate solution, water, brine, dried over sodium sulfate and evaporated in vacuo to obtain crude side chain (1). The crude product was purified by silica gel column chromatography to obtain pure side chain (0.61 g) with an overall yield of 56% in four steps. The product was characterized by ¹H NMR and ¹³C NMR. The chiral purity was found to be 98.6% by chiral GC with a chemical purity of 97%.

### Example 3: Chiral Selectivity in Enantioselective Reduction of Aryl Alkynyl Ketone Enzymatic Enantioselective Reduction of Aryl Alkynyl Ketone (16)

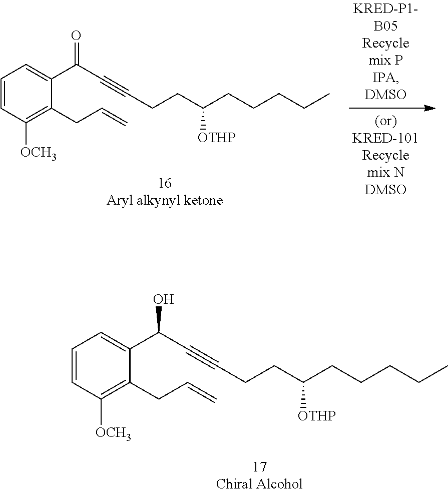

### Method A

To KRED enzyme KRED-P1-B05 (10 mg) was added KRED Recycle Mix P (from Codexis) (475 μL). A solution of aryl alkynyl ketone (16) (2 mg) in 25 μL DMSO was added. This was stirred at 30° C. for 24 h. At this stage the reaction was quenched by adding acetonitrile. The reaction mixture was centrifuged, and the supernatant was separated and extracted with MTBE. The organic layer was evaporated in vacuo to obtain crude chiral alcohol (17) (2 mg). The chiral purity by HPLC was found to be >99.0%.

### Method B

To KRED enzyme KRED-101 (10 mg) was added KRED Recycle Mix N (from Codexis) (475 μL). A solution of aryl alkynyl ketone (16) (2 mg) in 25 μL DMSO was added. This was stirred at 30° C. for 24 h. At this stage the reaction was quenched by adding acetonitrile. The reaction mixture was centrifuged, and the supernatant was separated and extracted with MTBE. The organic layer was evaporated in vacuo to obtain crude chiral alcohol (17) (2 mg). The chiral purity by HPLC was found to be >98.5%.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A method of synthesizing a compound of formula (1) 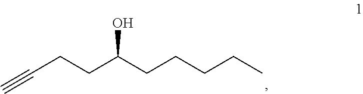
the method comprising:
reacting a compound of formula (8) 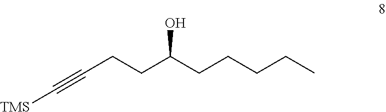
with 3,5 dinitrobenzoyl
chloride to form a compound of formula (9) 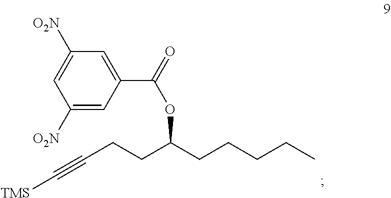 [SMILES: CCCCC[C@@H](CCC#C[Si](C)(C)C)OC(=O)c1cc([N+](=O)[O-])cc([N+](=O)[O-])c1]
and
deprotecting the compound of formula (9) to form the compound of formula (1) 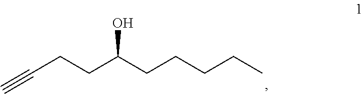
wherein TMS is trimethylsilyl.

2. The method of claim 1, wherein said deprotecting is performed in the presence of a base.

3. The method of claim 2, wherein said base is NaOH.

4. The method of claim 1, further comprising
(A) reacting a compound of formula (5) 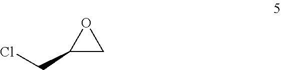
with butylmagnesium chloride to form a compound of formula (6) 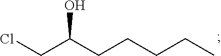
(B) reacting the compound of formula (6) with a base to form a compound of formula (7) 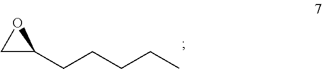
and
(C) reacting the compound of formula (7) with trimethylsilyl propyne to form the compound of formula (8).

5. The method of claim 4, wherein said reacting (A) is performed in the presence of CuI.

6. The method of claim 4, wherein the base in said reacting (B) is NaOH.

7. The method of claim 1, wherein said reacting (C) is performed at a temperature from −60° to −70° C.

8. A method of synthesizing treprostinil comprising:
protecting the compound of formula (1) synthesized according to the method of claim 1 to form a compound of formula (3) 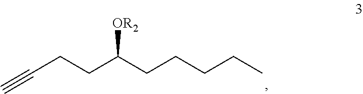
wherein R² is a hydroxy protecting group;
reacting the compound of formula (3) with a compound of formula (2) 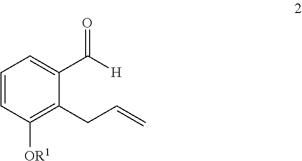
to form a compound of formula (15) 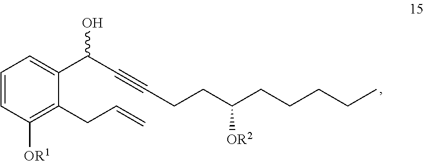
wherein R′ is a hydroxy protecting group; and
converting the compound of formula (15) in treprostinil (4)

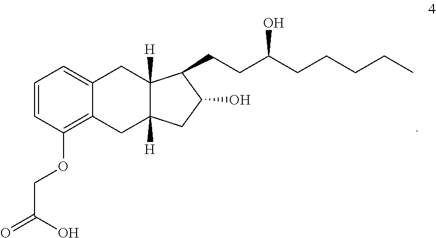 [SMILES: CCCCC[C@H](O)CC[C@@H]1[C@H]2Cc3cccc(OCC(=O)O)c3C[C@H]2C[C@H]1O]

9. A method of synthesizing a compound of formula (1) 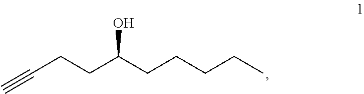
the method comprising:
reacting a compound of formula (10) 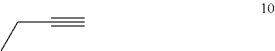
with a compound of formula (11) 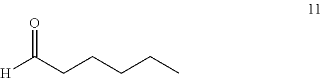
to form a compound of formula (12) 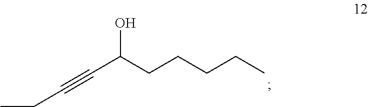
oxidizing the compound of formula (12) to form a compound of formula (13) 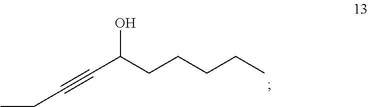
enantioselectively reducing the compound of formula (13) to form a chiral compound of formula (14) 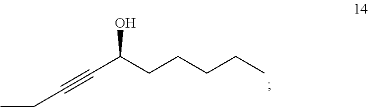
and
isomerizing the compound of formula (14) to form the compound of formula (1)

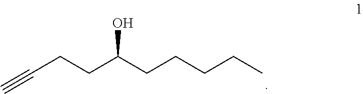

10. The method of claim 9, wherein said reacting the compound of formula (10) with the compound of formula (11) is performed in the presence of BuLi.

11. (canceled)

12. The method of claim 9, wherein said oxidizing is performed in the presence of an oxidation catalyst.

13. (canceled)

14. The method of claim 9, wherein said reducing is performed in the presence of an enantioselective catalyst.

15. The method of claim 14, wherein the enantioselective catalyst is a metal containing catalyst.

16. The method of claim 15, wherein the metal containing catalyst is a ruthenium containing catalyst.

17. (canceled)

18. The method of claim 14, wherein the enantioselective catalyst is an enzyme catalyst.

19. The method of claim 18, wherein the enzyme catalyst is a ketoreductase.

20. (canceled)

21. The method of claim 9, wherein said isomerizing is performed in the presence of a zipper isomerization reagent.

22. (canceled)

23. A method of synthesizing treprostinil comprising:
protecting the compound of formula (1) synthesized according to the method of claim 9 to form a compound of formula (3) 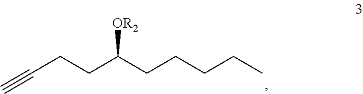
wherein R² is a hydroxy protecting group;
reacting the compound of formula (3) with a compound of formula (2) 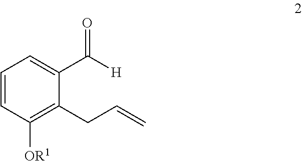
to form a compound of formula (15) 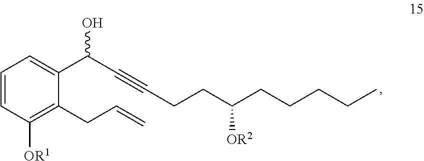
wherein R¹ is a hydroxy protecting group —(CH₂)ₙX; X being H, phenyl, —CN, —OR³ or COOR³; n being 1 to 3, R³ being an alkyl, a hydroxy protecting group or a substituted or unsubstituted benzyl group; and
converting the compound of formula (15) into treprostinil (4)

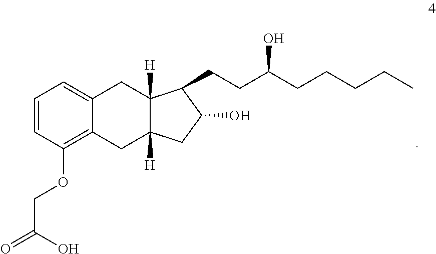 [SMILES: CCCCC[C@H](O)CC[C@@H]1[C@H]2Cc3cccc(OCC(=O)O)c3C[C@H]2C[C@H]1O]

24. A method of synthesizing treprostinil comprising:
enantioselectively reducing a compound of formula (16) 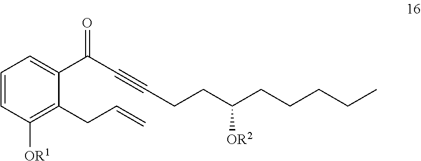
to form a compound of formula (17) 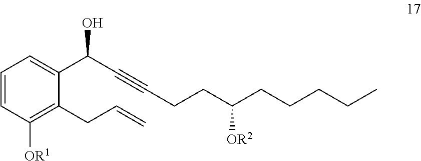
in the presence of an enantioselective enzyme catalyst; and
converting the compound of formula (17) into treprostinil (4) 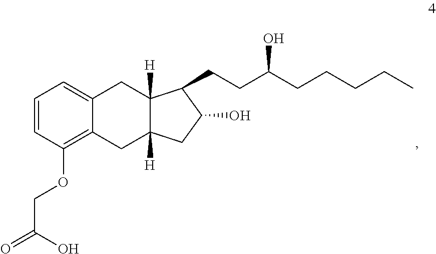
wherein R¹ is a hydroxy protecting group —(CH₂)ₙX; X being H, phenyl, —CN, —OR³ or COOR³; n being 1 to 3, R³ being an alkyl, a hydroxy protecting group or a substituted or unsubstituted benzyl group; and R² is a hydroxy protecting group.

25-30. (canceled)

31. A treprostinil batch synthesized according to the method of claim 24.

32-34. (canceled)
